(19) United States
(12) Patent Application Publication
(10) Pub. No.: US 2010/0004565 A1
(43) Pub. Date: Jan. 7, 2010

(54) SENSOR ARRANGEMENT FOR HOME REHABILITATION

(75) Inventors: Gerd Lanfermann, Aachen (DE); Edwin Gerardus Johannus Maria Bongers, Roermond (NL); Juergen Te Vrugt, Aachen (DE); Richard Daniel Willmann, Siegburg (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/519,839

(22) PCT Filed: Dec. 18, 2007
(86) PCT No.: PCT/IB07/55189

§ 371 Date: Jun. 18, 2009

(30) Foreign Application Priority Data
Dec. 21, 2006 (EP) 06126897.5

(51) Int. Cl. A61B 5/103

(52) U.S. Cl. 600/587

(57) ABSTRACT

A sensor arrangement for home rehabilitation in particular after a stroke comprising at least two sensors. The sensors are attached to the user's body. Each sensor includes—a receiver for receiving a first signal being generated from a source outside the sensor 10 arrangement—a sensor processing unit processing the first signal and initializing a second signal upon reception of the first signal, the second signal including information regarding the identity of the sensor—a transmitter for transmitting the second signal to a central processing unit 15 On receipt of the first and/or the second signal a time stamp is generated for each sensor for a determination of the location of each sensor relative to a source of the first signal trough comparison of the different time stamps.

The present invention relates to a system and a method for rehabilitation and/or physical therapy for the treatment of neuromotor disorders, such as a stroke.

After a stroke patients often suffer of disturbances in movement coordination. These disturbances are the least well understood but often the most debilitating with respect to functional recovery following brain injury. These deficits in coordination are expressed in the form of abnormal muscle synergies and result in limited and stereotype movement patterns that are functionally disabling. The result of these constraints in muscle synergies is for example an abnormal coupling between shoulder abduction and elbow flexion in the arm, which significantly reduces a stroke survivor reaching space when he/she lifts up the weight of the impaired arm against gravity. Current neurotherapeutic approaches to mitigate these abnormal synergies have produced limited functional recovery. In the leg the expression of abnormal synergies results in coupling hip/knee extension with hip adduction. The result of this is a reduced ability of activating hip abductor muscles in the impaired leg during stance.

One of the most prominent disabilities stroke survivors suffer from is half sided paralysis of the upper limbs. Rehabilitation exercises are proven to be efficient in regaining motor control, provided the training is intense and the patient is guided in the therapy.

When traditional therapy is provided in a hospital or rehabilitation center, the patient is usually seen for half-hour sessions, once or twice a day. This is decreased to once or twice a week in outpatient therapy.

Current studies indicate that motor exercising for improving the coordination of the patient can be done at home as part of a tele-rehabilitation solution. Technical solutions for unsupervised home stroke rehabilitation require the use of markers or sensors for acquiring the patient's posture during exercises. Posture acquisition by sensors is an attractive and much explored option.

The problem with such an approach is that existing marker or sensor-based tracking systems assume the user to be skilled enough to place selected sensors on specific locations on user's body typically the hand, lower arm and upper arm; thus consistent results should be achieved. The sensors are therefore labeled, colored or enumerated.

This assumption becomes unrealistic, if the user is a stroke victim and is suffering from cognitive impairment. In this case instead, the exact position of the markers on the limbs will differ from one use to the other, since the user is not able to fix the marker or sensor in exactly the same position because of a loss of control of the movement of his arms hands and/or fingers. The necessary differentiation of the various sensors and the assignment according to their placement is an additional burden for a stroke victim.

It is therefore an object of the present invention to minimize the amount of instructions for placing the sensors on a user's body and therefore to facilitate the fastening procedure.

This object is solved by a sensor arrangement for home rehabilitation in particular after a stroke comprising at least two sensors to be attached to user's body, each sensor including a receiver for receiving a first signal being generated from a source outside the sensor arrangement, a sensor processing unit processing the first signal and initializing a second signal upon reception of the first signal, the second signal including information regarding the identity of the sensor, a transmitter for transmitting the second signal to a central processing unit, wherein upon receipt of the first and/or the second signal a time stamp is generated for each sensor to determine the location of each sensor relative to the source of the first signal through comparison of the different time stamps.

With this the user is allowed to take any sensor and attach it to any required body part without caring, which sensor has to be put on which part of the body.

After the placement of all sensors on the different body parts, the distance of each sensor relative to the source of the first signal is calculated to determine the location of each sensor. Several different ways can be established to achieve this calculation.

A sensor arrangement according to a first embodiment therefore includes a central processing unit which comprises a time stamp generator wherein a time stamp is generated in the central processing unit every time a second signal has been received. The time stamp then is stored in the central processing unit together with information regarding the identity of the sensor.

In an alternative embodiment each sensor comprises a time stamp generator, the time stamp generators of all sensors being synchronized with each other, wherein a time stamp is generated and stored in the sensor processing unit immediately after receipt of the first signal. In this embodiment each sensor comprises a time stamp generator. Thus the risk of missing a second signal at the central processing unit is not essential, since it is possible to interrogate the time stamps being stored in the sensor processing unit any time it is desired and this interrogation can be replicated as often as wanted.

The first and/or the second signal may be an acoustic signal and/or an impulse signal and/or a light signal. The first signal may for example be generated in that the user extends both arms and claps his hands. With this an acoustic signal as well as an impulse signal is generated which both may be detected by the sensors depending on whether the sensor is an acoustic sensor or an impulse sensor or both. It is also possible to use electronic or mechanical devices to trigger a sound or another signal. These devices may be included in the central processing unit.

Since the first signal should be a signal which enables to determine the location of the sensors relatively to each other on a human's body a sound signal or an impulse signal is favorable nowadays since there has to be a time lag between the detection of the first signal at two neighboring sensors and the progress of the sound waves or the impulse is relatively slow so that it may easily be detected.

In case of the measurement of the impulse generated by clapping the user's hands the measuring can be conducted without caring whether the arms are straightened with the user's arms since the impulse progresses along the arms anyway.

In case a light signal is used as a first signal the difference between two time stamps being generated at two neighboring sensors upon reception of the first signal would be very little so that the measuring means has to be very accurate to detect the difference in timestamps. Existing measuring means which are able to detect a measurable result even if a light signal is used presently are very cost intensive. In the future if the costs for such sensing means is not as expensive anymore it might make sense to also take light signals as first signals.

Using a light signal as second signal on the other hand makes sense since the spectrum of light is very brought and easy to determine and there is only a very short delay between its emitting from the sensors and its determination by the central processing unit more precisely a photosensitive device, being provided in the central processing unit in one embodiment.

In one embodiment of the present invention, the first signal may be generated in that the user extends both arms and claps his hands. With this an acoustic signal as well as an impulse signal is generated which both may be detected by the sensors depending on whether the sensor is an acoustic sensor or an impulse sensor.

To be sure, that the body parts on which the sensors have been placed are aligned with the source of the first signal the central processing unit may comprise a communication means to issue instructions, how to proceed with the initializing of the sensor arrangement and to advise the user how to position himself relative to the source of the first signal to get a proper measurement. If the sensors are to be placed on a user's arms the user may be instructed by the central processing unit to first place for example three sensors on his left arm and afterwards to clap his hands. After the determination of the sensors relative to each other on the left arm the central processing unit may instruct the user to put the other sensors on his right arm and afterwards to clap his hands again or instead of clapping his hands to indicate in any way that he finished the placement of the sensors and is ready for the identification procedure of the sensors for example by saying “placement finished” or by pushing a button so that a first signal from an external source may be generated thereupon.

The external source may be an electronic or mechanical device to trigger a sound or another signal as a first signal. These devices may also be included in the central processing unit.

To avoid a generation of a second signal because of signals from the surrounding not being generated on purpose but only by accident, the sensor processing unit may compare the signals received by the receiver with a predetermined signal stored in the sensor processing unit corresponding to the first signal, wherein a second signal is only generated by the sensor processing unit if the signal being received by the receiver matches with the stored signal. This can be realized by using a microphone as receiver which registers the passing of for example sound waves, wherein the sensor processing unit may check if the detected sound waves correspond to the predetermined signal or is due to other noises, e.g. by checking the spectrum, pulse height and or width.

In one embodiment each sensor may emit different upon receipt of the first signal to enable the central processing unit to simultaneously determine the identity of the sensor generating the second signal while receiving the second signal. Therefore, each sensor may generate a second signal, which simultaneously shows its identity. This can be achieved for example if the second signal is a light signal and each sensor emits with a different wave length characterizing its identity. The light signals may be detected by a photosensitive device being provided in the central processing unit. In the central processing unit each wave length may be dedicated to a specific sensor. Thus, the timestamp of each sensor relative to the source of the first signal can be stored easily in the central processing unit together with the identity of the sensor generating the second signal, by comparing the timestamps and thereby accurately determine which sensor has been reached by the first signal at first, second or third. The same applies if a second signal is an auditive signal, for example if the frequency is chosen specific for each sensor.

The sensors may exchange their time stamps with each other in an all-to-all communication to determine their relative position to the source of the first signal. Of course, it is also possible that after the determination of the relative position to the source of the first signal the sensors may change their identification among each other by completing an ordering process after exchanging of their time stamps so that their identity afterwards matches with the order of placement on the user's body for example from wrist to shoulder.

The sensors of the sensor arrangement according to the invention may each be fixed on carriers to be fastened to a user's body wherein the carriers may be formed in a way, that they ensure a minimal distance between two neighboring sensors which is necessary to get a difference in the measurement of the time stamps because of the progression of the first signal.

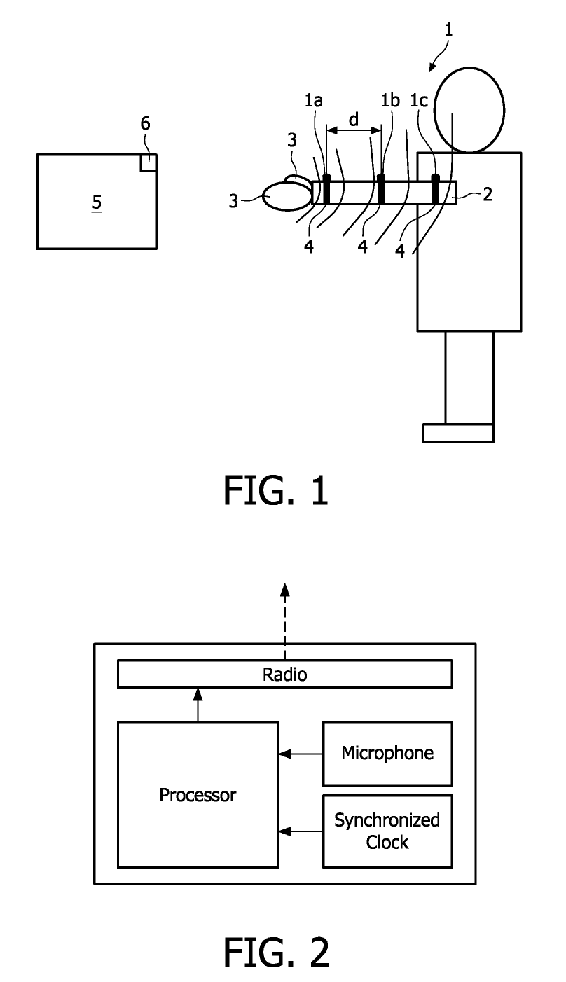

A sensor arrangement for home rehabilitation in particular after a stroke which meets the above-mentioned object and provides other beneficial features in accordance with the presently preferred exemplary embodiment of the invention will be described below with reference to FIGS. 1 and 2.

Those familiar with the state-of-the-art will readily appreciate that the description given herein with respect to those figures is for explanatory purposes only and is not intended to limit the scope of the invention.

FIG. 1 shows a sensor arrangement according to the invention using the passing of a sound signal; and

FIG. 2 shows an example of a sensor of such an arrangement, wherein a time stamp being generated in the sensor.

FIG. 1 shows a sensor arrangement 1 for home rehabilitation in particular after a stroke.

To minimize the amount of instructions for placing the sensors 1a, 1b, 1c on the user's arms 2 and therefore to facilitate the fastening procedure the sensor arrangement provides a sensor detecting and identification mechanism, which enables an assignment of the sensors 1a, 1b, 1c regarding their placement on the user's body by calculating the distance of the sensors 1a, 1b, 1c relative to a source of a first signal for example a sound signal which in a first embodiment is generated by the user clapping his hands 3.

The sensor arrangement 1 consists of six separate sensors, three of them 1a, 1b, 1c being attached to a user's left arm 2 and three of them (not shown) being placed on the user's right arm (also not shown).

Each sensor 1a, 1b, 1c is fixed on a carrier 4 the carrier being is fastened one after another to the user's arms 2 without following a special order. In this embodiment three equivalent sensors 1a, 1b, 1c are attached at a user's hand 3, lower arm and upper arm 3a and trunk 3b. The carrier 4 is build in a way that there is a minimal distance d between each carrier 4 to ensure a minimal distance between two neighboring sensors 1a and 1b or 1b and 1c which is necessary to get a difference in time stamps being measured between the sensors 1a, 1b, 1c because of the progression of a first signal being indicated in FIG. 1 by the curves at the user's left arm 2. The minimal distance d between two sensors 1a, 1b, 1c may be calculated be the rate of the progression of the first signal proportional to the resolution of the time stamp generator.

As shown in FIG. 2 for the calculation of the distance of the sensors 1a, 1b, 1c relative to the users hands 3 each sensor 1a, 1b, 1c includes a receiver for receiving the first signal, a sensor processing unit for processing the first signal and initializing a second signal upon a reception of the first signal as well as an transmitter for transmitting the second signal to a central processing unit 5.

In this embodiment each sensor 1a, 1b, 1c furthermore comprises a time stamp generator for example a clock, wherein the clocks in all sensors 1a, 1b, 1c and the sensors not shown are synchronized with each other thus enabling a determination of the location of each sensor 1a, 1b, 1c relative to the hands of the user trough comparison of the different timestamps in combination with their identities.

The method of determining of the sensor position works as follows:

First, the user is invited by the central processing unit 5 to attach any three sensors 1a, 1b, 1c to his left arm without caring of the sequencing or the identity or the sensors 1a, 1b, 1c. Afterwards the user is instructed to extend both arms and to clap his hands 3. Alternatively, it is also possible that the user need not to clap his hands 3 but only indicates the central processing unit 5 that he finished the placement procedure and he is prepared to continue with the position determining of the sensors 1a, 1b, 1c. Afterwards an electronic or mechanical device within the central processing unit 5 invites the user to extend his arms 2 in a direction of the source of the first signal. Afterwards the central processing unit 5 triggers a sound or an impulse or something adequate as a first signal.

The step of extending the users arms 2 in direction of the source of the first signal may be neglected if the first signal is an impulse signal for example the clapping of the user's hands 3 since the impulse signal progresses inside the users arms 2 and therefore bended arms would not change the measuring result considerably.

Every time a first signal is received by the receiver, here a microphone which registers the passing sound waves, and transmitted to the sensor processing unit the sensor processing unit compares the signals received by the microphone with a predetermined signal stored in the sensor processing unit. Only if the received signal corresponds to the stored signal in spectrum, pulse height and/or width, a time stamp is generated by the time stamp generator and stored in the sensor processing unit to avoid a generation of a time stamp because of noise from the surrounding not matching the predetermined signal.

Simultaneously or short afterwards a second signal is generated by the sensor processing unit and transferred to the central processing unit 5 by the transmitter, in this embodiment a radio, immediately after receipt of the first signal and the generation of the timestamp, the second signal including information regarding the identity of the sensor 1a, 1b, 1c as well as its corresponding timestamp.

The central processing unit 5 receives the time stamps and identities of all sensors 1a, 1b, 1c. The comparison of the different time stamps leads to an information which sensors 1a, 1b, 1c has been placed to which specific body part for example the sensor 1a with “identity 2” is on the left hand, the sensor 1b with “identity 1” is on the left lower arm and the sensor 1c with “identity 3” is on the left upper arm.

As soon as all sensors 1a, 1b, 1c have been identified the user is informed that the determining of the sensors 1a, 1b, 1c on the left arm 2 has been finished and that the same measurement now has to be accomplished for the right arm. In the next step all left arm sensors 1a, 1b, 1c will not take part in this measurement because they have already been localized.

Thus the sensor arrangement 1 enables the user to take any sensor 1a, 1b, 1c and attach it to any body part to be monitored for example the left arm 2 without the need of taking care of which sensor 1a, 1b, 1c has to be put on which part of the body and of the sequencing of the sensors 1a, 1b, 1c.

If the time stamps are inconsistent for example because the measure time difference between to sensors 1a, 1b, 1c is too long or too short this indicates that either not all sensors 1a, 1b, 1c had been placed correctly or the user is not aligned to the source of the first signal properly. In this case the user is asked to check the sensor positioning and the positioning of his arms 2 relative to the central processing unit 5 again and than to generate the first signal again for example by clapping his hands.

In an alternative embodiment (not shown) the central processing unit 5 comprises a central time stamp generator wherein a time stamp is generated in the central processing unit 5 each time a second signal has been transmitted from the sensor arrangement 1 to the central processing unit 5 together with information regarding the identity of the sensor 1a, 1b, 1c emitting this second signal. To distinguish the sensor 1a, 1b, 1c which generated and transmitted the second signal immediately or simultaneously with the reception of the second signal in one embodiment each sensor 1a, 1b, 1c replies upon reception of the first signal in a very distinguished way, that means for example if the second signal being generated by the sensors 1a, 1b, 1c is a light signal each sensor may emit with the different wave length characterizing its identity. The light signals in this case are detected by a photosensitive device 6 being provided in the central processing unit 5 and afterwards processed in the processing unit 5. In the processing unit 5 each different wave length is dedicated to a specific sensor 1a, 1b, 1c so that right away with the reception of the signal it is possible to address the time stamp generated upon reception to the right sensor 1a, 1b, 1c. This makes it possible, to accurately determine which sensor 1a, 1b, 1c has been reached by the first signal at first, second or third on the left arm.

In a further embodiment the sensors 1a, 1b, 1c may exchange their time stamps with each other in an all-two-all-communication to determine their relative position to the source of the first signal. Afterwards, each sensor 1a, 1b, 1c may do the ordering process individually. By this each sensor 1a, 1b, 1c knows about its relative position from the source of the sound, here the central processing unit 5. If information on the sensor placement is available on the sensor 1a, 1b, 1c, each sensor 1a, 1b, 1c knows its supporting body part. This is of importance if significant parts of the sensor data processing is done on the sensor 1a, 1b, 1c and not on the central processing unit 5.

Of course, it is also possible that after the determination of the relative position to the source of the first signal the sensors 1a, 1b, 1c may change their identity among each other by completing an ordering process after exchanging of their time stamps so that their identity afterwards matches with the order of placement on the user's body for example from wrist to shoulder.

1. A sensor arrangement (1) for home rehabilitation in particular after a stroke comprising at least two sensors (1a, 1b, 1c) to be attached to a user's body, each sensor (1a, 1b, 1c) including:
a receiver for receiving a first signal being generated from a source outside the sensor arrangement (1);
a sensor processing unit processing the first signal and initializing a second signal upon reception of the first signal, the second signal including information regarding the identity of the sensor (1a, 1b, 1c)
a transmitter for transmitting the second signal to a central processing unit (5) wherein upon receipt of the first and/or the second signal a time stamp is generated for each sensor (1a, 1b, 1c) for a determination of the location of each sensor (1a, 1b, 1c) relative to a source of the first signal trough comparison of the different time stamps.

2. A sensor arrangement (1) according to claim 1, characterized in that the central processing unit (5) comprises a time stamp generator and a time stamp is generated in the central processing unit (5) each time a second signal has been received each time stamp being separately stored in the central processing unit (5) together with information regarding the identity of the sensor (1a, 1b, 1c).

3. A sensor arrangement (1) according to claim 1, characterized in that each sensor (1a, 1b, 1c) comprises a time stamp generator, the time stamp generators being synchronized with each other, wherein a time stamp is generated and stored in the sensor processing unit immediately after receipt of a first signal.

4. A sensor arrangement (1) according to claim 1, characterized in that the first and/or the second signal is an acoustic signal and/or an impulse signal and/or a light signal.

5. A sensor arrangement (1) according to claim 1, characterized in that the sensor processing unit compares the signals received by the receiver with a predetermined signal corresponding to the first signal, wherein a second signal is only generated by the sensor processing unit if the signal being received by the receiver matches with the predetermined signal.

6. A sensor arrangement (1) according to claim 1, characterized in that the central processing unit (5) comprises a communication means to issue instructions how to proceed with the initializing of the sensor arrangement (5).

7. Sensor arrangement (1) according to claim 1, characterized in that each sensor (1a, 1b, 1c) generates a different second signal upon receipt of the first signal to thereby simultaneously indicating its identity to the central processing unit (5).

8. Sensor arrangement (1) according to claim 1, characterized in that the sensors (1a, 1b, 1c) exchange their time stamps with each other in an all-to-all communication to determine their position relative to the source of the first signal.

9. A sensor arrangement (1) according to claim 8, characterized in that the sensors (1a, 1b, 1c) complete an ordering process after exchangement of their time stamps.

10. A sensor arrangement (1) according to claim 1, characterized in that the sensors (1a, 1b, 1c) comprise carriers, which are formed in a way that a minimal distance between each other for an execution of the determination of their position is maintained.

11. Method for determining of a sensor position on a users limb by a sensor arrangement (1) according to claim 1 including the steps of
Placing at least two sensors (1a, 1b, 1c) on one limb of the users body without caring of the sequencing of the sensors (1a, 1b, 1c);
Extending the limb (2) being equipped with the sensors (1a, 1b, 1c) towards the source of the first signal;
Generating a first signal to be detected by the sensors (1a, 1b, 1c);
Processing the first signal in the sensor processing unit and initializing a second signal upon reception of the first signal, the second signal including information regarding the identity of the sensor(1a, 1b, 1c);
Generating a time stamp by the time stamp generator if the first and/or the second signal corresponds to a pre specified signal stored in the sensor processing unit in spectrum, pulse and/or width; and
Comparing the different time stamps to evaluate the position of the sensors (1a, 1b, 1c) relative to the source of the first signal.

12. Method for determining of the sensor position according to claim 11 characterized in that the first signal is generated in that the user is invited to extend both arms (2) and to clap his hands (3).

13. Method for determining of a sensor position according to claim 11, characterized in that the time stamps are generated in each sensor.

14. Method for determining a sensor position to claim 11 characterized in that the time stamps are generated in the sensor processing unit.
